(19) United States
(12) Patent Application Publication
(10) Pub. No.: US 2011/0295192 A1
(43) Pub. Date: Dec. 1, 2011

(54) HOLLOW NEEDLE FOR AN OPHTHALMIC SURGICAL INSTRUMENT

(76) Inventor: Volker Geuder, Heidelberg (DE)

(21) Appl. No.: 12/993,002

(22) PCT Filed: Mar. 31, 2009
(86) PCT No.: PCT/DE09/00407

§ 371 Date: Feb. 2, 2011

(30) Foreign Application Priority Data
May 16, 2008 (DE) 10 2008 023 967.4

(51) Int. Cl. A61F 9/007

(52) U.S. Cl. 604/22

(57) ABSTRACT

A hollow needle for an ophthalmic surgical instrument for the in-vivo fragmentation of organic lenses by way of ultrasound, comprising a connecting region (1) for coupling to the instrument and a work region (3) configured at a free end (2), said work region having an effective surface (4) for emitting ultrasonic waves, a suction channel (5) for suctioning out lens fragments extending through the hollow needle, said channel being open in the work region (3) and the opening (6) thereof being formed or delimited by the effective surface (4), characterized in that the suction channel (5) has at least one further opening (7), which acts as a bypass and is configured in the work region (3).

The invention concerns a hollow needle for an ophthalmic surgical instrument for in vivo fragmentation of organic lenses by means of ultrasound with a connection area for connection to the instrument and a work area formed on the free end with an effective surface for emission of ultrasonic waves, in which a suction channel open in the work area for suction of lens fragments extends through the hollow needle, whose opening is formed or delimited by the effective surface.

A generic hollow needle is known from DE 196 46 881 C1. Specifically, this is a hollow needle for an ophthalmic surgical instrument for in vivo fragmentation of lenses by high frequency activation of the hollow needle, the hollow needle simultaneously serving for suction of lenses through an internal suction channel. The hollow needle includes an annular end that forms the opening of the suction channel.

Ultrasound-operated hollow needles of the generic type are used in cataract operations in ophthalmic surgery. The free end of the hollow needle is placed in high-frequency axial movement and brought directly against the cataract. Ultrasonic waves are emitted from the annular end to emulsify the tissue. Separated lens parts or lens fragments are withdrawn together through the hollow needle with a rinsing fluid supplied to the eye.

To amplify the emitted ultrasonic field, it is already known to tooth the face side or free end of the hollow needle, i.e., the effective surface, in order to increase the effective surface by the toothing. The effective surface that serves for emission of ultrasonic waves is enlarged on this account, so that the efficiency of the instrument or the hollow needle is improved.

The hollow needle known from DE 196 46 881 C 1, however, is problematical in practice, since larger lens fragments are not rarely withdrawn, which lead to at least temporary clogging in the area of the suction opening. There is also the hazard that the surgeon, with the opening of the suction channel, will get too close to the area of material not to be withdrawn, so that damage to the tissue could occur, because of the unduly large partial vacuum that develops. If one were to increase the suction pressure in the ophthalmic surgical instrument, one could counteract undesired clogging in the front area of the suction channel, but more extensive damage or injury would have to be tolerated.

The underlying task of the present invention is therefore to configure and modify a hollow needle for an ophthalmic surgical instrument of the generic type, so that clogging in the front area of the suction channel is effectively avoided, while avoiding undesired injuries.

The aforementioned task is solved according to the invention by a hollow needle for an ophthalmic surgical instrument for in vivo fragmentation of organic lenses by means of ultrasound with the features of claim 1. According to it, the generic hollow needle is characterized by the fact that the suction channel has at least one additional opening acting as a bypass, which is formed in the work area.

It was recognized according to the invention that clogging in the work area of the hollow needle can be avoided by a simple design expedient, namely, by forming an additional opening in the work area, to be understood as a bypass with reference to the flow path. In other words, in addition to the front suction opening in the work area, a further opening is provided, through which air can be introduced in the case of suction of larger lens fragments, so that further drawing in of the lens fragment and therefore more extensive clogging from the partial vacuum developing in the suction channel is avoided. If one assumes that most ophthalmic surgical instruments can generate pulsating partial vacuums and at least also brief overpressures in the suction channel, a lens fragment not completely drawn into the hollow needle can be ejected again without effort, which is not or is no longer the case in the event of already occurred clogging. In any event, the additional opening avoids unduly large partial vacuum in the suction channel, which already in itself counteracts clogging of the suction channel.

As already mentioned, at least one additional opening in the sense of a bypass is provided. Advantageously, several additional openings can be provided along the periphery of the work area, in which case it is particularly advantageous, if the additional opening or additional openings is or are formed close to the effective surface. The configuration of the additional openings close to the effective surface is a quite particular advantage to the extent that undesired clogging of the suction channel is counteracted precisely there, in which case further along the suction channel, the partial vacuum required to withdraw the reduced lens fragments can be built up. If the additional openings were formed farther from the effective surface, the suction effect of the hollow needle would be substantially reduced. This is also effectively avoided according to the invention.

It should be mentioned here that at least one additional opening is provided, in which case several additional openings can be formed along the periphery of the work area. In the interest of simplicity, only “the additional opening” will be discussed subsequently.

The additional opening can discharge in the work surface and therefore open outward at the work surface. This precaution has the advantage that not only is a bypass created by this expedient, but the work surface is significantly enlarged, which again favors emission of ultrasonic waves or the ultrasonic field.

The additional opening can be designed as a passage, especially a hole, milling, notch, etc., in the wall of the work area of the hollow needle. Production of the passage by a laser technique is also conceivable and an advantage, especially in view of the miniaturized configuration of the work area. Electroerosive methods to produce the passage are also conceivable.

The additional opening or passage can have any shape. Advantageously, it is formed as a slit in the wall of the work area of the hollow needle. It can be designed angled, oval, round or otherwise. In any event, it is essential that it be dimensioned in size or area, so that a sufficiently large partial vacuum is formed in the work area of the hollow needle, regardless of the passage, but firm suction of oversized lens fragments is effectively avoided.

Also advantageously, the work area is radially widened relative to the main body of the hollow needle. In other words, the hollow needle has a radially widened work area, preferably designed cylindrical. The work area of the hollow needle therefore forms a sort of suction bell, which, on the end of the work area, is reduced to the actual inside diameter of the hollow needle or the diameter of the suction channel. Between the work area and the reduced suction channel, one or several steps can be provided, which promote the size reduction process in the interior of the work area, because a sharp-edged or scalloped design.

Also advantageously, the work area ends with a beveled surface, which forms the effective surface, and whose front end is understood to be the tip of the hollow needle. Any angles of the beveled surface relative to the longitudinal axis can be implemented, in order to also enlarge the effective surface in so doing.

The additional openings advantageously form in the area of the effective surface facing way from the front end or tip, especially since this area forms the actual entry into the suction channel and a sort or “emergency ventilation” in the sense of a bypass is significant there. In the case of provision of additional openings, these are also formed in the area of the effective surface facing away from the front end or tip and optionally in the side areas of the effective surface, namely, from the aforementioned standpoint.

In principle, it is conceivable that the effective surface is formed in one plane, preferably smooth. It is also conceivable that the effective surface is made corrugated, so that the effective surface is also enlarged.

The effective surface could also be stepped, scalloped or toothed as a further advantage.

With quite particular advantage, the effective surface includes indentations or notches formed equidistant to each other, which interrupt the actual effective surface along its periphery. According to this embodiment, the actual effective surface consists of individual segments, which can be designed triangular or polygonal.

The teeth, scallops, notches, etc. can be designed, so that the front area of the outer wall is repeatedly opened relative to the actual effective surface, so that several additional openings are formed on this account, which interrupt the actual effective surface. In other words, the outer wall of the work area is interrupted (outward), so that emergency ventilation occurs through the side openings in the event of approach or even touching of the actual effective surface. Through this expedient, the development of an unduly high partial vacuum in the interior of the work area is effectively avoided.

With reference to production of the hollow needle, it is advantageous to make it in one piece, in which case it can consist of titanium or a titanium alloy.

There are now different possibilities for advantageously embodying and modifying the instructions of the present invention. For this purpose, the claims subordinate to claim 1, on the one hand, and the following explanation of two preferred practical examples of the invention with reference to the drawing, on the other, are referred to. In conjunction with explanation of the preferred practical examples of the invention with reference to the drawing, preferred embodiments and modifications of the instructions are also explained in general. In the drawing

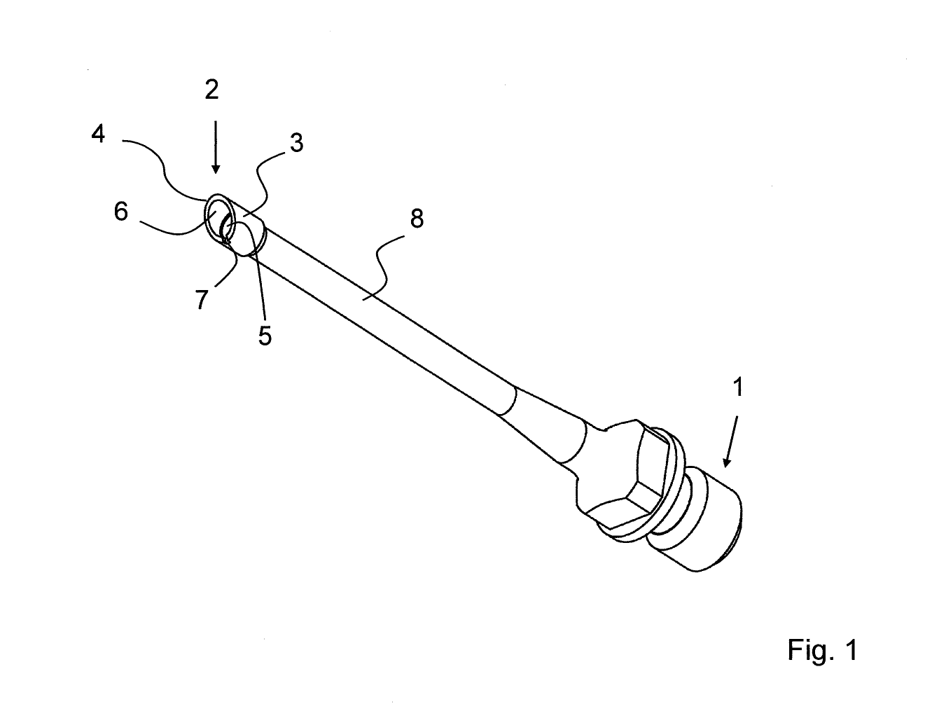

FIG. 1 shows a schematic view of a first practical example of a hollow needle according to the invention with an additional opening in the work area,

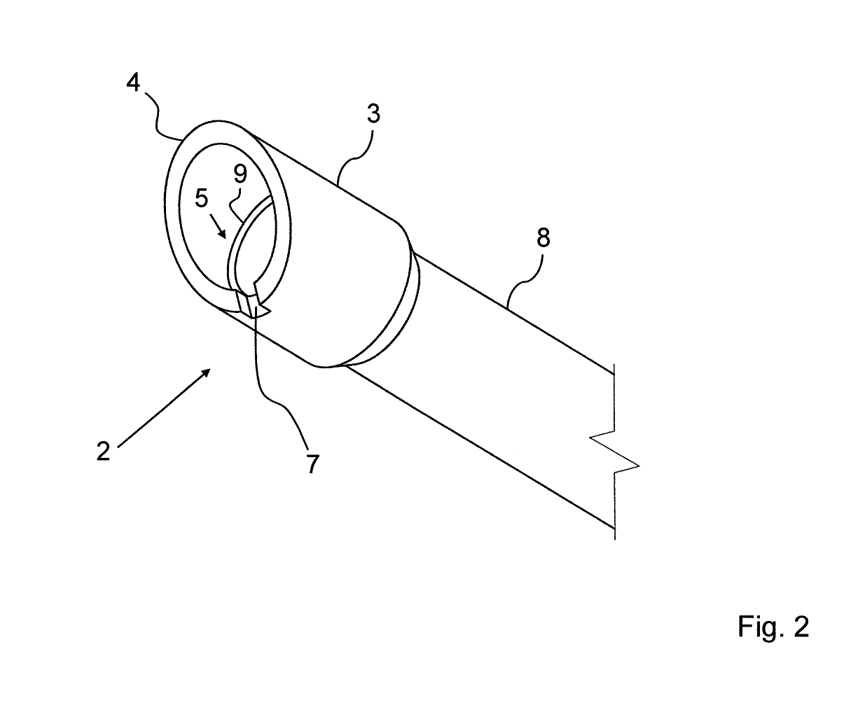

FIG. 2 shows a schematic view, enlarged, of the work area of the hollow needle from FIG. 1,

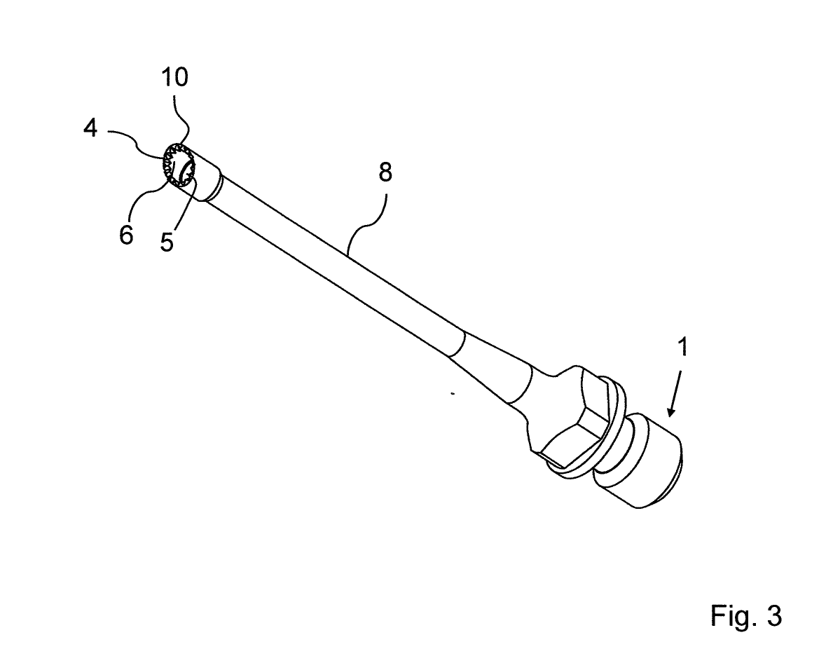

FIG. 3 shows a schematic view of a second practical example of a hollow needle according to the invention with several additional openings in the sense of a scalloped and outward opened work area, and

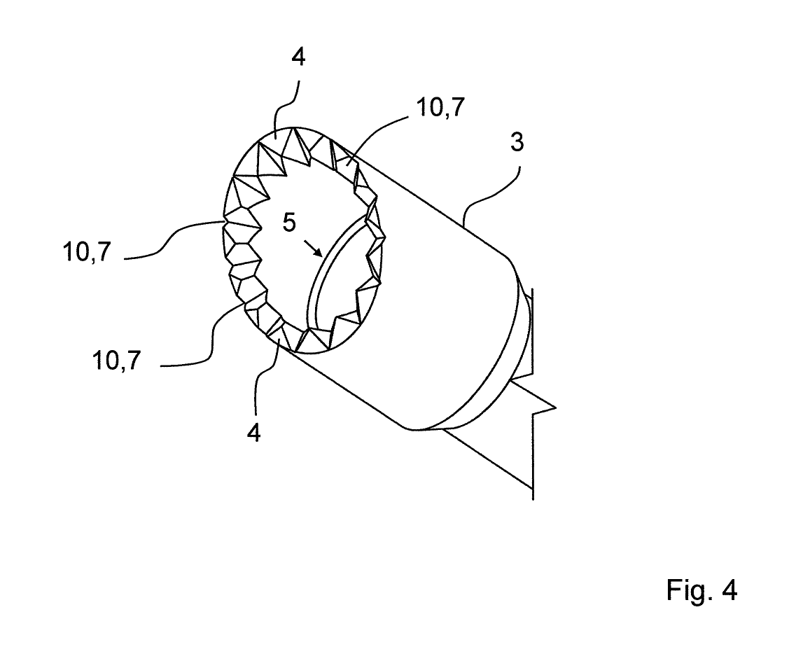

FIG. 4 shows a schematic view, enlarged, of the work area of the hollow needle from FIG. 3.

FIG. 1 shows a first practical example of the hollow needle according to the invention for an ophthalmic surgical instrument for in vitro fragmentation of organic lenses by means of ultrasound. The hollow needle includes a connection area 1 for connection to the instrument not shown in the figures and a work area 3 formed on free end 2. The work area 3 is for emission of ultrasonic waves, in which a suction channel 5 open in the work area 3 extends through the hollow needle for suction of lens fragments. The opening 6 of the suction channel 5 is formed or delimited by the effective surface 4.

According to the invention, the suction channel 5 has at least one additional opening 7 acting as bypass, which is formed in work area 3.

In the practical example depicted in FIGS. 1 and 2, a single additional opening 7 is provided, in which case several such openings 7 can also advantageously be provided.

The additional opening 7 is formed in the vicinity of the effective surface 4, namely, as a rectangular passage. The additional opening 7 ends in the effective surface 4 and is therefore open to the effective surface 4. Because of this expedient, the additional opening 7 significantly enlarges the effective surface 4. FIGS. 1 and 2 also show that the work area 3 is radially widened relative to the main body 8 of the needle, namely, to a radially widened cylindrical work area 3. The free end of the work area 3 is beveled and defined by the effective surface 4.

FIGS. 1 and 2 also clearly show that in the interior of the work area 3, a step 9 or at least a beveled transition area is formed, which favors a size reduction of the lens material.

It should also be noted that both the outer edge and the inner edge of the work area 3, i.e., the inner and outer limitation of effective surface 4, can be designed sharp-edged. Beveling, both outward and inward, of the effective surface 4 would favor emission of ultrasonic waves.

In the practical example depicted in FIGS. 3 and 4, the effective surface 4 is interrupted by equidistant notches 10, in which case the notches 10 also interrupt or slit the front area of the outer surface of work area 3. Through this expedient, several additional openings 7 are created along the periphery of the radially widened work area 3. At the same time, the effective surface is simultaneously enlarged inward, namely, through notches 10.

With reference to the features that cannot be deduced from the figures, the general part of the description is referred to, to avoid repetitions.

Finally, it is explicitly pointed out that the practical examples of the hollow needle according to the invention just described serve merely to explain the claimed instructions, but do not limit them to the practical examples.

### LIST OF REFERENCE NUMBERS

1 Connection area
2 Free end
3 Work area
4 Effective surface
5 Suction channel
6 Opening (suction opening)
7 Additional opening (bypass)
8 Main body of the hollow needle
9 Step (in the interior of the work area)
10 Notches (additional opening)

1.-18. (canceled)

19. A hollow needle for an ophthalmic surgical instrument for in vitro fragmentation of organic lenses by means of ultrasound, said hollow needle comprising:
a connection area for connection to the instrument; and
a work area formed on a free end with an effective surface for emission of ultrasonic waves, in which a suction channel opening the work area extends through the hollow needle for suction of lens fragments, whose opening is formed or delimited by the effective surface,
wherein the suction channel has at least one additional opening acting as bypass, which is formed in the work area.

20. The hollow needle according to claim 19, wherein several additional openings are provided.

21. The hollow needle according to claim 19, wherein the additional opening(s) is (are) formed near the effective surface.

22. The hollow needle according to one of the claim 19, wherein the additional opening(s) discharges/discharge in work area and therefore increases/increase the effective surface.

23. The hollow needle according to claim 19, wherein the additional opening is designed as a passage, especially a hole, milling, etc., in a wall of the work area of the needle.

24. The hollow needle according to claim 19, wherein the additional opening is designed as a passage, especially as a slit, in a wall of the work area of the needle.

25. The hollow needle according to claim 24, wherein the passage is angled, oval or round.

26. The hollow needle according to o claim 19, wherein the work area is radially widened relative to a main body of the needle.

27. The hollow needle according to claim 26, wherein the work area is designed cylindrical.

28. The hollow needle according to claim 19, wherein the work area ends with a beveled surface that forms the effective surface, and whose front end forms a tip.

29. The hollow needle according to claim 28, wherein the additional opening is formed in an area of the effective surface facing away from the front end or tip.

30. The hollow needle according to claim 28, wherein the additional openings are formed in an area of the effective surface facing away from the front end or tip and optionally in side areas of the effective surface.

31. The hollow needle according to claim 19, wherein the effective surface is formed in one plane, preferably smooth.

32. The hollow needle according to claim 19, wherein the effective surface is designed corrugated.

33. The hollow needle according to claim 19, wherein the effective surface is designed scalloped or toothed.

34. The hollow needle according to claim 19, wherein the effective surface has indentations or notches equidistant from each other, which interrupt the actual effective surface along the periphery.

35. The hollow needle according to claim 33, wherein teeth, scallops, notches, etc. are formed, so that a front area of an outer wall is repeatedly opened relative to the actual effective surface, so that several additional openings are formed on this account, which interrupt the actual effective surface.

36. The hollow needle according to claim 19, wherein the hollow needle has a one-piece design, preferably from titanium or a titanium alloy.
